(19) United States
(12) Patent Application Publication
(10) Pub. No.: US 2012/0233742 A1
(43) Pub. Date: Sep. 20, 2012

(54) Strapless ear muffs

(76) Inventors: Rickie Ellen Wood-Bovee, Ponce De Leon, FL (US); Tzong Jiunn Wu, Miramar Beach, FL (US)

(21) Appl. No.: 13/065,062

(22) Filed: Mar. 14, 2011

(51) Int. Cl. A61F 11/14

(52) U.S. Cl. 2/209

(57) ABSTRACT

This invention solves the dilemma of those who cannot or will not wear conventional ear protection because it causes compression pain or headache due to the overhead strap or compression fitting on the ear. The Strapless Ear Muffs fit over the ear and provide cold weather protection without the overhead strap or pinching on the ear. They are lightweight and are not held on by compression.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

This invention was created to fill a needed gap in clothing accessories and protection from the cold.

All other ear muffs provide cover and protection by clamping a covering over the ears held in place by a spring type strap over the head. This style can create discomfort and even headache.

Another variety of ear covering provides a singular cover that lightly pinches over the ear creating discomfort for some wearers.

Our intent was to provide warmth and protection by creating a cover that did not clamp or pinch.

### SUMMARY OF THE INVENTION

Having a background in clothing and accessory construction and design as well as having participated in many cold weather outdoor activities such as cross-country skiing, mountain climbing, and wilderness survival, the need for ultimate protection from the cold, especially to the extremities, is understood.

Within that understanding, given the increasing cold weather being experienced in the United States, especially in the Northern regions, it is our intent to provide a means of protecting the ears without the nuisance of the over-head band.

In our experience with the ear muffs available, which use an over-the-head band to secure them, we suffered from pain and headache after wearing them for only a short time. The STRAPLESS EAR MUFFS can be worn for an extended period of time without discomfort. As an accessory they can look fashionable and be functional as well.

Knowing that trapped air is the best insulator, our STRAPLESS EAR MUFFS provide the necessary insulation by using the lightweight, air trapping, foam.

### BRIEF DESCRIPTION OF THE SEVERAL VIEWS OF THE DRAWING

FIG. 1—A view of the Strapless Ear Muff showing the inside surface and the ear shaped cutout

FIG. 2—A view of the inside surface that lies against the head; the plastic reinforcement (1) and the ear cutout (2).

FIG. 3—Side view showing the laminated layers of foam: Outside layer, ½″ medium density foam; Middle layer, ¾″ medium density foam; Third layer, ⅛″ heavy density craft foam; Plastic reinforcement, 1/16″ plastic.

FIG. 4—Cross section showing how the layers, laminated together, create the cavity that protects the ear. Reference 1 indicates the cavity.

FIG. 5—Interior view depicting the cavity without the inside surface. The outside and middle layer of ½″ medium density foam. Reference 1—outside layer of foam viewed from the inside. Reference 2—middle layer of foam, creating the cavity, viewed from inside.

FIG. 6—Complete Outside Fabric Cover, constructed of faux fur or fleece, removable and held on with ¼″ elastic.

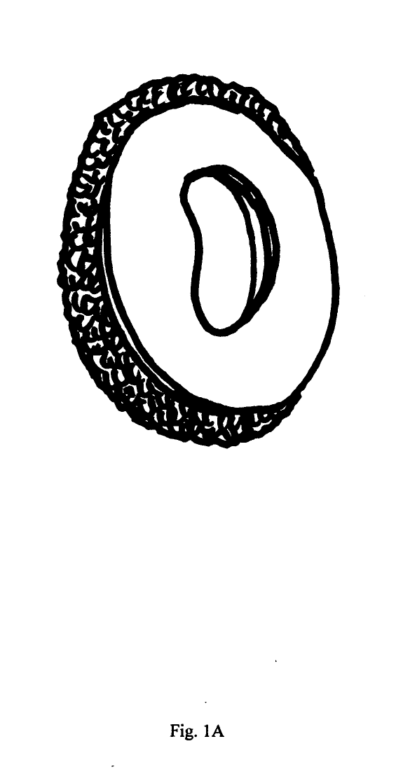

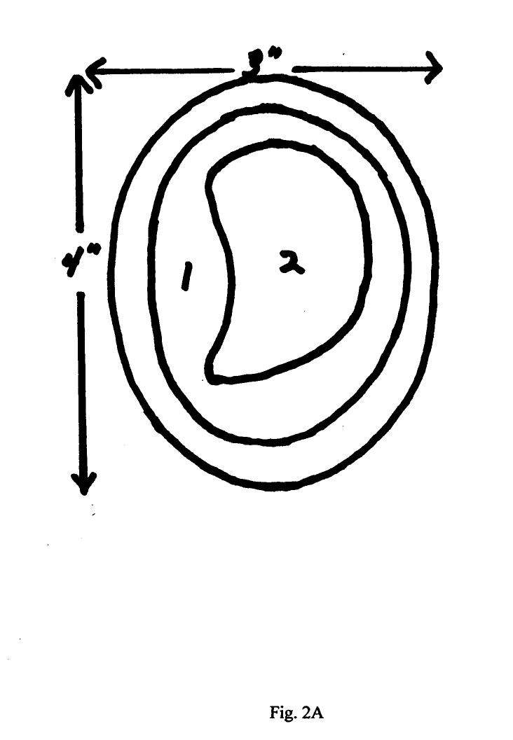

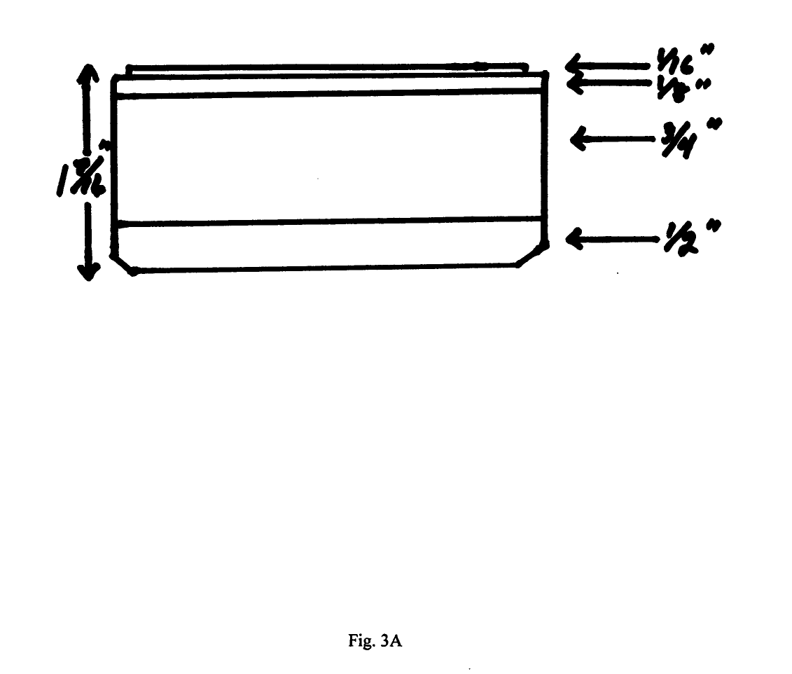

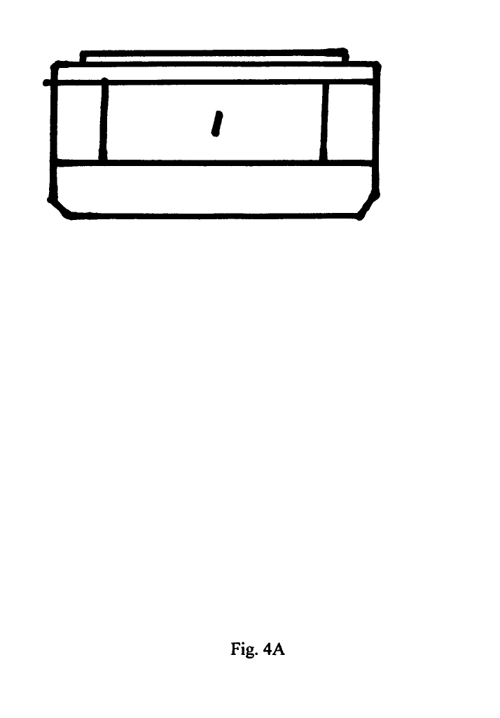

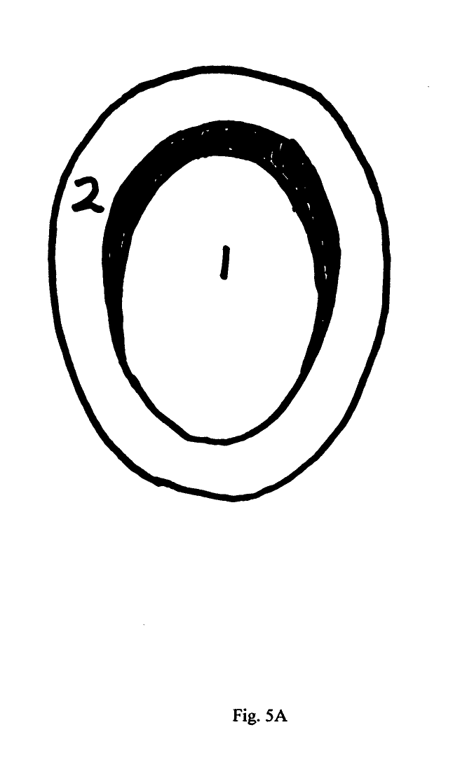

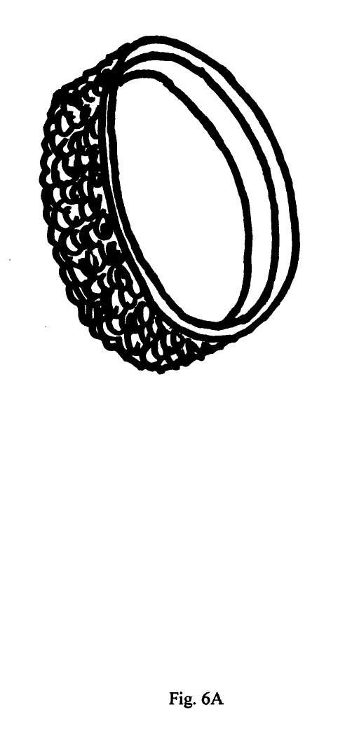

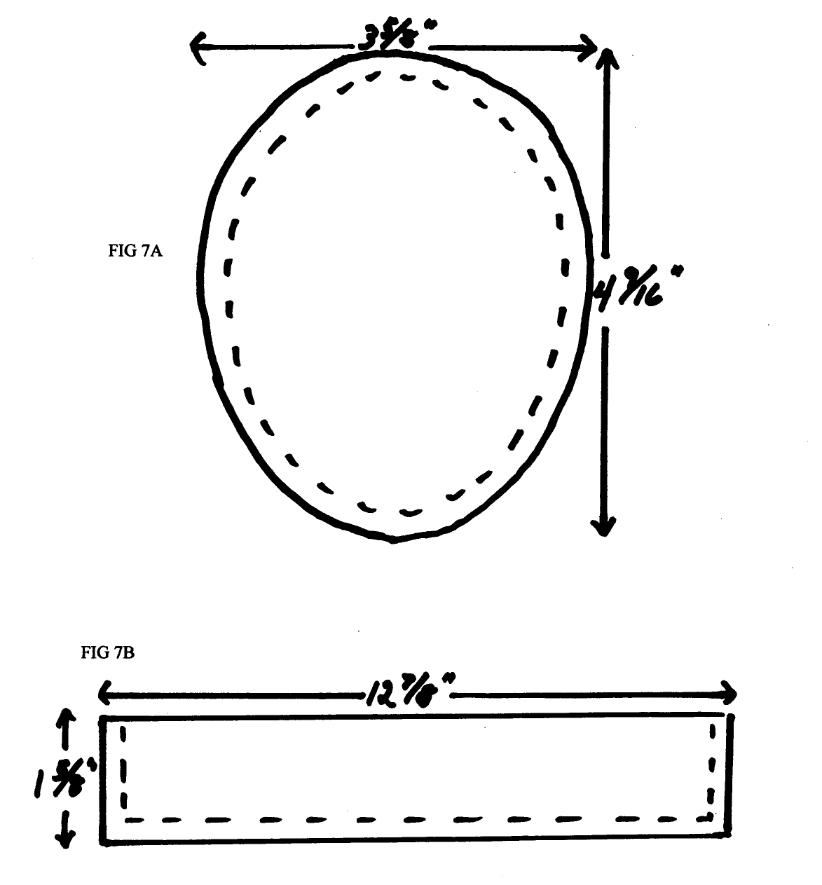

FIG. 7A—Outside Fabric Cover, Fabric piece with 1/4″ seam allowance.
7B—Outside Fabric cover, side band with seam allowances shown

### DETAILED DESCRIPTION OF THE INVENTION

The STRAPLESS EAR MUFFS provide a warm, lightweight atmosphere for the ears in inclement weather. The ear muffs are designed so that the wearer places them on by fitting the ear in through the ear shaped opening thereby placing the ear inside a cavity, surrounded by medium density foam. The air trapped by the foam and the fabric covering provides maximum insulation and warmth

The ear muffs are created by cutting and laminating pieces of ½″ medium density foam in such a manner as to create a cavity in which the ear is placed and then, surrounded by the foam and fabric covering, is protected from severe temperatures.

The cavity is created by cutting a strip of foam ½″×¾″ by 11″. That piece is then laminated to the outside piece, in a circular manner, creating the cavity.

1- The strapless ear muffs provide a comfortable and lightweight fit as well as protection from the cold without pressure to the head from an over-the-head strap, which can, in some people, cause discomfort or even headache.

2- The medium density foam, from which the body of the ear muff is made, traps air which is the best insulator and protector from cold temperatures. The foam body is then covered with fleece or faux fur, which provides additional insulation.
